(19) United States
(12) Patent Application Publication
(10) Pub. No.: US 2022/0273603 A1
(43) Pub. Date: Sep. 1, 2022

(54) METHOD FOR TREATING HYPERHIDROSIS

(71) Applicant: HISAMITSU PHARMACEUTICAL CO., INC., Tsukuba-shi (JP)

(72) Inventors: Kana IMAMURA, Tsukuba-shi (JP); Naoko FUJITA, Tsukuba-shi (JP); Yasunari MICHINAKA, Tsukuba-shi (JP)

(21) Appl. No.: 17/682,205

(22) Filed: Feb. 28, 2022

Related U.S. Application Data
(63) Continuation-in-part of application No. 16/079,208, filed on Aug. 23, 2018.

(30) Foreign Application Priority Data
Feb. 25, 2016 (JP) 2016-034620

(51) Int. Cl. A61K 31/222; A61K 9/00; A61K 47/10; A61K 47/02

(52) U.S. Cl. CPC A61K 31/222; A61K 9/0014; A61K 47/10; A61K 47/02

(57) ABSTRACT

A method for treating hyperhidrosis is disclosed. The method comprises administering a liquid topical preparation to a patient in need thereof. The liquid topical preparation comprises water and oxybutynin or a pharmaceutically acceptable salt thereof. The oxybutynin or pharmaceutically acceptable salt thereof is present in a range of, for example, from 10 mass % to 20 mass % based on a total mass of the liquid topical preparation.

### TECHNICAL FIELD

The present subject matter relates to a method for treating hyperhidrosis.

### BACKGROUND ART

Methods that involve administering a topical composition comprising an anticholinergic drug such as oxybutynin have been proposed as methods for treating hyperhidrosis (Patent Literature 1 and Patent Literature 2).

### CITATION LIST

### Patent Literature

Patent Literature 1: U.S. Patent Application Publication No. 2014/0037713
Patent Literature 2: International Publication No. WO 2007/046102

### SUMMARY OF PRESENT SUBJECT MATTER

### Technical Problem

A liquid topical preparation comprising oxybutynin for treating hyperhidrosis has not yet been marketed as a product, and therefore, a desirable dosage and administration for the liquid topical preparation has not been found.

### Solution to Problem

As a result of intensive studies to solve the above problem, the present inventors have found a liquid topical preparation that exhibits a sufficient sweat suppressing effect, and have completed the present subject matter.

Specifically, the present subject matter provides a method for treating hyperhidrosis comprising administering to a patient in need thereof, a liquid topical preparation, comprising: water; and oxybutynin or a pharmaceutically acceptable salt thereof; wherein the oxybutynin or pharmaceutically acceptable salt thereof is present in a range of from 10 mass % to 20 mass % based on a total mass of the liquid topical preparation. The liquid topical preparation may be administered to the patient once daily. The liquid topical preparation may be administered to both of the patient's palms. The oxybutynin or pharmaceutically acceptable salt thereof may comprise oxybutynin hydrochloride. The oxybutynin or pharmaceutically acceptable salt thereof may be present at about 20 mass % based on a total mass of the liquid topical preparation. The liquid topical preparation may be administered in the form of a lotion. The liquid topical preparation may further comprise at least one ingredient to enhance accumulation of the oxybutynin or pharmaceutically acceptable salt thereof in the patient. The at least one ingredient to enhance accumulation may comprise at least one salt selected from the group consisting of lactate, tartrate, acetate, and phosphate. The at least one salt may include sodium lactate. The liquid topical formulation may further comprise ethanol. The oxybutynin or pharmaceutically acceptable salt thereof may comprise oxybutynin hydrochloride and the liquid topical preparation may further comprise sodium lactate and ethanol. The liquid topical preparation may be administered in the form of a lotion. The liquid topical preparation may be administered once daily to both of the patient's palms, for at least 10 days, or alternatively at least 15 days, or at least 20 days, or at least 25 days, or at least 30 days. Alternatively, the liquid topical preparation may be administered for at least 4 weeks, or for at least 2 weeks, or for at least 3 weeks, or for at least 5 weeks, or for at least 6 weeks, or for at least 7 weeks, or for at least 8 weeks. The administration may reduce sweat produced by the patient's palm by at least about 0.4 mg/cm²/minute, or alternatively at least about 0.1 mg/cm²/minute, or at least 0.2 mg/cm²/minute, or at least 0.3 mg/cm²/minute, or at least 0.5 mg/cm²/minute.

### BRIEF DESCRIPTION OF DRAWINGS

FIG. 1 is a graph showing the results of a test for examining the influence of salts in lotions on the accumulation of oxybutynin in porcine hair follicles.

FIG. 2 is a graph showing the results of a test for examining the influence of the concentrations of oxybutynin in lotions on the effect of suppressing sweating.

FIG. 3 is a graph showing the results of a test for examining the influence of the concentrations of oxybutynin in lotions on the effect of suppressing sweating.

FIG. 4 is a graph showing the results of a test for examining the influence of the concentrations of oxybutynin in lotions on the accumulation of oxybutynin in porcine hair follicles.

FIG. 5 is a graph showing the amount of change in the amount of sweat of a hyperhidrosis patient.

FIG. 6 is a graph showing the HDSS score of hyperhidrosis patients.

FIG. 7 is a graph showing changes in plasma concentration of oxybutynin.

### DESCRIPTION OF EMBODIMENTS

Hereinafter, the present subject matter will be described more specifically with reference to an embodiment.

In one embodiment, the liquid topical preparation to be administered to a patient in need thereof comprising water and an effective amount of oxybutynin or a pharmaceutically acceptable salt thereof, wherein the effective amount of oxybutynin or pharmaceutically acceptable salt thereof is present in a range of from 10 mass % to 20 mass % based on a total mass of the liquid topical preparation. In other embodiments, the oxybutynin or pharmaceutically acceptable salt thereof may be present in an amount of 10, 11, 12, 13, 14, 15, 16, 17, 18, 19, or 20 mass %, based on a total mass of the liquid topical preparation, or in any range of any two of these specifically enumerated amounts. The liquid topical preparation can be used for treating hyperhidrosis.

An example of a pharmaceutically acceptable salt of oxybutynin is oxybutynin hydrochloride.

The oxybutynin or pharmaceutically acceptable salt thereof is present in a range of from 10 mass % to 20 mass % based on a total mass of the liquid topical preparation. The oxybutynin or pharmaceutically acceptable salt thereof may be present in a range of from 15 mass % to 20 mass % based on the total mass of the liquid topical preparation. The lower limit of the oxybutynin or pharmaceutically acceptable salt thereof may be 10, 12, 15 or 18 mass % based on the total mass of the liquid topical preparation. The oxybutynin or pharmaceutically acceptable salt thereof may be present at about 20 mass % based on a total mass of the liquid topical preparation. In one embodiment oxybutynin or pharmaceutically acceptable salt thereof may be in a range of from 15 mass % to 25 mass %, or in a range of from 18 mass % to 22 mass % based on a total mass of the liquid topical preparation.

The liquid topical preparation may further comprise a dicarboxylic acid ester. The dicarboxylic acid ester decreases the viscosity of the liquid topical preparation, thereby suppressing “stickiness.” Specific examples of the dicarboxylic acid ester include diisopropyl adipate, diethyl sebacate, diisopropyl sebacate, dimethyl succinate, dibutyl adipate, diisobutyl adipate, dioctyl adipate, dioctyl sebacate, diethyl phthalate and dibutyl phthalate. The content of the dicarboxylic acid ester may range from 2.5 mass % to 15 mass % or 5 mass % to 15 mass % based on the total mass of the liquid topical preparation. The lower limit of the content of the dicarboxylic acid ester may be 1, 2.5, 3, 3.75, 5 or 8 mass % based on the total mass of the liquid topical preparation. The upper limit of the content of the dicarboxylic acid ester may be 10, 11.25, 12 or 15 mass % based on the total mass of the liquid topical preparation. With an arbitrary combination of the lower limit and the upper limit of the content of the dicarboxylic acid ester, “stickiness” derived from the oxybutynin or pharmaceutically acceptable salt thereof can further be reduced. In one embodiment the dicarboxylic acid ester may be in a range of from 5 mass % to 10 mass %, or in a range of from 7.2 mass % to 8.8 mass % based on a total mass of the liquid topical preparation.

The mass ratio of the oxybutynin or pharmaceutically acceptable salt thereof to the dicarboxylic acid ester may range from 1:0.25 to 1:0.75. The lower limit of the mass ratio of the oxybutynin or pharmaceutically acceptable salt thereof to the dicarboxylic acid ester; that is, the lower limit of the mass of the oxybutynin or pharmaceutically acceptable salt thereof per unit mass of the dicarboxylic acid ester may be 1:0.75, 1:0.70, 1:0.65, 1:0.6, 1:0.55 or 1:0.5. The upper limit of the mass ratio of the oxybutynin or pharmaceutically acceptable salt thereof to the dicarboxylic acid ester; that is, the upper limit of the mass of the oxybutynin or pharmaceutically acceptable salt thereof g per unit mass of the dicarboxylic acid ester may be 1:0.05, 1:0.15, 1:0.25, 1:0.3, 1:0.33, 1:0.35 or 1:0.4. With an arbitrary combination of the lower limit and the upper limit of the mass ratio of the oxybutynin or pharmaceutically acceptable salt thereof to the dicarboxylic acid ester, “stickiness” derived from the oxybutynin or pharmaceutically acceptable salt thereof can further be reduced.

Water in the liquid topical preparation functions as medium for dissolving or dispersing the oxybutynin or pharmaceutically acceptable salt thereof as well as other components. The content of water may range from 10 mass % to 99 mass %, for example, based on the total mass of the liquid topical preparation. In one embodiment, water may be in a range of from 10 mass % to 40 mass %, in a range of from 20 mass % to 30 mass %, or in a range of from 24 mass % to 28 mass % based on a total mass of the liquid topical preparation.

The liquid topical preparation may further comprise at least one ingredient to enhance accumulation of the oxybutynin or pharmaceutically acceptable salt thereof in the patient, so as to enhance the accumulation of the oxybutynin or pharmaceutically acceptable salt thereof in skin appendages. Through enhancement of the accumulation, hyperhidrosis can be treated while suppressing side effects due to administration of the oxybutynin or pharmaceutically acceptable salt thereof such as xerostomia. The at least one ingredient to enhance accumulation comprises at least one salt selected from the group consisting of lactate, tartrate, acetate, and phosphate. The salt may be anhydride or hydrate. Lactic acid may be either L- or D-lactic acid, or may be an arbitrary mixture thereof. Tartaric acid may be any one of L-, D-, and meso-tartaric acid, or may be an arbitrary mixture thereof. Examples of the salt include a salt with a monovalent metal such as sodium, potassium and lithium, a salt with a divalent metal such as calcium and magnesium, a salt with a trivalent metal such as aluminum, and a salt with an amine compound such as ammonia, ethylenediamine, triethylamine, diethanolamine, triethanolamine and meglumine. From the viewpoint of improving the accumulation of the oxybutynin or pharmaceutically acceptable salt thereof in skin appendages, the salt is preferably lactate and more preferably sodium lactate.

The content of at least one ingredient to enhance accumulation of the oxybutynin or pharmaceutically acceptable salt thereof in the patient may range from, for example, 0.1 mass % to 10 mass % based on the total mass of the liquid topical preparation. In one embodiment, at least one ingredient to enhance accumulation of the oxybutynin or pharmaceutically acceptable salt thereof in the patient may be in a range of from 5 mass % to 10 mass %, or in a range of from 5.4 mass % to 6.6 mass % based on a total mass of the liquid topical preparation. The molar ratio of the oxybutynin or pharmaceutically acceptable salt thereof to at least one ingredient to enhance accumulation of the oxybutynin or pharmaceutically acceptable salt thereof in the patient in the liquid topical preparation may be, for example, within the range of 1:0.5 to 1:2.

The liquid topical preparation may comprise, in addition to the above components, one or more of any of the following: an additional solvent, an additional treating agent for the same or a different purpose, a lower alcohol, a surfactant, a preservation stabilizer, a fat, an oil, a solubilizer, a filler, a moisturizer, a pH regulating agent, an osmotic pressure regulator, a thickener, a refreshing agent, an astringent and a vasoconstrictor, for example, or any suitable pharmaceutical adjuvant or other suitable pharmaceutical ingredient.

The lower alcohol increases the solubility and dispersibility of the oxybutynin or pharmaceutically acceptable salt thereof, and increases the distributivity of the oxybutynin or pharmaceutically acceptable salt thereof into skin. Specific examples of the lower alcohol include methanol, ethanol and isopropanol. The content of the lower alcohol may range from, for example, 0 mass % to 90 mass % based on the total mass of the liquid topical preparation. In one embodiment, the lower alcohol may be in a range of from 10 mass % to 40 mass %, in a range of from 30 mass % to 50 mass %, or in a range of from 36 mass % to 44 mass % based on a total mass of the liquid topical preparation.

The surfactant is useful for emulsifying the oxybutynin or pharmaceutically acceptable salt thereof in a medium such as water. Specific examples of the surfactant include a nonionic surfactant (e.g., polysorbate 20, polysorbate 80, polysorbate 60, polyoxyethylene hydrogenated castor oil 20, polyoxyethylene hydrogenated castor oil 40 and polyoxyethylene hydrogenated castor oil 60), an ionic surfactant and an amphoteric surfactant. The content of the surfactant may range from, for example, 0 mass % to 10 mass % based on the total mass of the liquid topical preparation.

Specific examples of the preservation stabilizer include paraben, isopropylmethylphenol, phenoxyethanol and thymol.

Specific examples of the fat or oil and the solubilizer include a fatty acid and a fatty alcohol.

Specific examples of the filler include an inorganic powder (e.g., talc, montmorillonite, smectite and kaolin) and an organic powder.

Specific examples of the moisturizer include a polyhydric alcohol, saccharides, urea, a vaseline and a paraffin.

The liquid topical preparation can have a pH within the range of 4.5 to 7.5. pH determination is performed by any commonly-accepted method in the pharmaceutical industry, such as for example by using a composite glass electrode in accordance with “2.54 pH Determination” in General Tests, Processes and Apparatus, the Japanese Pharmacopoeia, Sixteenth Edition.

The liquid topical preparation may be in a form of lotion or liniment, for example, or in a form of embrocation or spray, for example, contained in an appropriate container (for example, a spray container for spraying the liquid preparation, a container for applying the liquid preparation and an aerosol container).

The liquid topical preparation can be manufactured by mixing thoroughly the above components.

After the container is shaken as necessary to thoroughly mix the components homogeneously, the liquid topical preparation is applied to, sprinkled on or sprayed on the areas of skin where sweating should be suppressed, and is spread as needed.

In one embodiment, the liquid topical preparation is administered to a human adult once a day. The administration site is preferably a site where the amount of sweat is high, for example, the palm. The timing of administration is not limited, and can be, for example, at bedtime. The amount of oxybutynin or pharmaceutically acceptable salt thereof in the liquid topical preparation per administration is 60 mg to 150 mg, preferably 70 mg to 120 mg, more preferably 80 mg to 110 mg, and particularly preferably 90 mg to 100 mg. The volume of per administration per administration may be 400 μL to 600 μL, and is preferably 450 μL to 550 μL. The density of the liquid topical preparation may be 0.90 g/cm³ to 1.0 g/cm³. A mean plasma concentration of oxybutynin after repeating administration of the liquid topical preparation once a day for 8 weeks to a human adult may be, for example, 15 ng/mL to 25 ng/mL, and a mean plasma concentration of N-desethyloxybutynin, an active metabolite, may be, for example, 4.5 ng/mL to 8 ng/mL. In this case, a ratio of mean plasma concentration of oxybutynin/mean plasma concentration of N-desethyloxybutynin may be, for example, 1.875 to 5.556. Within such a concentration range and/or ratio, the liquid topical preparation typically exhibits a sufficient sweat suppressing effect.

In one embodiment, the liquid topical preparation is administered to a human adult once a day. The administration site is preferably a site where the amount of sweat is high, for example, the palm. The timing of administration is not limited, and can be, for example, at bedtime. The amount of oxybutynin or pharmaceutically acceptable salt thereof in the liquid topical preparation per administration is 90 mg to 100 mg. The volume of per administration per administration may be 400 μL to 600 μL, and is preferably 450 μL to 550 μL. The density of the liquid topical preparation may be 0.90 g/cm³ to 1.0 g/cm³.

When the liquid topical preparation is administered to a human adult in a single administration, a maximum plasma concentration of oxybutynin can be, for example, 0.8 ng/mL to 40 ng/mL, a time required to reach the maximum plasma concentration can be, for example, 10 hours to 24 hours, and an area under a plasma drug concentration-time curve of oxybutynin from a time of administration to a final time of concentration measurement can be, for example, 9 ng h/mL to 291 ng·h/mL. In this case, a plasma concentration of oxybutynin at 24 hours after the administration (C₂₄) is preferably 0.7 ng/mL to 12 ng/mL. The liquid topical preparation showing above pharmacokinetic parameters exhibits a sufficient sweat suppressing effect.

When the liquid topical preparation is administered to a human adult in a single administration, a ratio of a maximum plasma concentration of N-desethyloxybutynin to a maximum plasma concentration of oxybutynin (maximum plasma concentration of N-desethyloxybutynin/maximum plasma concentration of oxybutynin) is, for example, 0.04 to 0.55, and a ratio of an area under a plasma drug concentration-time curve of N-desethyloxybutynin from a time of administration to a final time of concentration measurement to an area under a plasma drug concentration-time curve of oxybutynin from a time of administration to a final time of concentration measurement (area under a plasma drug concentration-time curve of N-desethyloxybutynin from a time of administration to a final time of concentration measurement/area under a plasma drug concentration-time curve of oxybutynin from a time of administration to a final time of concentration measurement) is, for example, 0.06 to 0.31. In this case, a ratio of a plasma concentration (C₂₄) of N-desethyloxybutynin to a plasma concentration (C₂₄) of oxybutynin at 24 hours after the administration (C₂₄ of N-desethyloxybutynin/C₂₄ of oxybutynin) is preferably 0.1 to 0.6. The liquid topical preparation showing above pharmacokinetic parameters exhibits a sufficient sweat suppressing effect.

When the liquid topical preparation is administered to a human adult once a day repeatedly and a steady-state is reached, a maximum plasma concentration (Cₘₐₓ) of oxybutynin is, for example, 5 ng/mL to 46 ng/mL, a time (tₘₐₓ) required to reach the maximum plasma concentration is, for example, 1 hour to 22 hours, an area under a plasma drug concentration-time curve of oxybutynin (AUC₀₋₂₄) is, for example, 75 ng h/mL to 610 ng h/mL, and a half-life (t_{1/2}) of a plasma concentration of oxybutynin is, for example, 10 hours to 90 hours. In this case, a plasma concentration (C₂₄) of oxybutynin at 24 hours after the administration is preferably 2 ng/mL to 44 ng/mL, a minimum plasma concentration (Cₘᵢₙ) of oxybutynin is preferably 2 ng/mL to 17 ng/mL, an apparent clearance (CL/F) in the route of administration other than intravenous administration calculated from the area under the plasma drug concentration-time curve (AUC₀₋₂₄) at the administration interval of oxybutynin is preferably 140 L/h to 1150 L/h. The liquid topical preparation showing above pharmacokinetic parameters exhibits a sufficient sweat suppressing effect.

When the liquid topical preparation is administered to a human adult once a day repeatedly and a steady-state is reached, a ratio of a maximum plasma concentration (Cₘₐₓ) of N-desethyloxybutynin to a maximum plasma concentration (Cₘₐₓ) of oxybutynin (Cₘₐₓ of N-desethyloxybutynin/Cₘₐₓ of oxybutynin) is, for example, 0.08 to 0.46; a ratio of an area under a plasma drug concentration-time curve (AUC₀₋₂₄) of N-desethyloxybutynin in an administration interval to an area under a plasma drug concentration-time curve (AUC₀₋₂₄) of oxybutynin in an administration interval (AUC₀₋₂₄ of N-desethyloxybutynin in an administration interval/AUC₀₋₂₄ of oxybutynin in an administration interval) is, for example, 0.15 to 0.65. In this case, a ratio of a plasma concentration (C₂₄) of N-desethyloxybutynin to a plasma concentration (C₂₄) of oxybutynin at 24 hours after the administration (C₂₄ of N-desethyloxybutynin/C₂₄ of oxybutynin) is preferably 0.18 to 1.5. The liquid topical preparation showing above pharmacokinetic parameters exhibits a sufficient sweat suppressing effect.

When the liquid topical preparation is administered to a human adult once a day repeatedly, a ratio of a maximum plasma concentration (Cₘₐₓ) of oxybutynin at a steady-state to a maximum plasma concentration (Cₘₐₓ) of oxybutynin at a first administration (Cₘₐₓ at a steady-state/Cₘₐₓ at a first administration) is preferably 0.5 to 13.5, a ratio of an area under a plasma drug concentration-time curve (AUC₀₋₂₄) of oxybutynin in an administration interval at a steady-state to an area under a plasma drug concentration-time curve (AUC₀₋₂₄) of oxybutynin from a first administration to 24 hours later (AUC₀₋₂₄ at a steady-state/AUC₀₋₂₄ at a first administration) is preferably 1 to 32, a ratio of a maximum plasma concentration (Cₘₐₓ) of N-desethyloxybutynin at a steady-state to a maximum plasma concentration (Cₘₐₓ) of N-desethyloxybutynin at a first administration (Cₘₐₓ at a steady-state/Cₘₐₓ at a first administration) is preferably 1.4 to 17, a ratio of an area under a plasma drug concentration-time curve (AUC₀₋₂₄) of N-desethyloxybutynin in an administration interval at a steady-state to an area under a plasma drug concentration-time curve (AUC₀₋₂₄) of N-desethyloxybutynin from a first administration to 24 hours later (AUC₀₋₂₄ at a steady-state/AUC₀₋₂₄ at a first administration) is preferably 2 to 45. The liquid topical preparation showing above pharmacokinetic parameters exhibits a sufficient sweat suppressing effect.

In one embodiment, the liquid topical preparation is administered to a patient in need thereof once daily at a dose of 90 mg to 100 mg of the oxybutynin or pharmaceutically acceptable salt thereof and the liquid topical preparation exhibits at least one pharmacokinetic parameter of the following (a) to (I):
(a) a maximum plasma concentration of oxybutynin in a single administration of the liquid topical preparation is 0.8 ng/mL to 40 ng/mL, and a time required to reach the maximum plasma concentration is 10 hours to 24 hours;
(b) an area under a plasma drug concentration-time curve of oxybutynin from a time of administration to a final time of concentration measurement in a single administration of the liquid topical preparation is 9 ng·h/mL to 291 ng·h/mL;
(c) a maximum plasma concentration of oxybutynin is 5 ng/mL to 46 ng/mL when a steady-state is reached by repeating administration of the liquid topical preparation once a day, and a time required to reach the maximum plasma concentration is 1 hour to 22 hours;
(d) an area under a plasma drug concentration-time curve of oxybutynin in an administration interval when a steady-state is reached by repeating administration of the liquid topical preparation once a day is 75 ng·h/mL to 610 n·gh/mL;
(e) a half-life of a plasma concentration of oxybutynin when a steady state is reached by repeating administration of the liquid topical preparation once a day is 10 hours to 90 hours;
(f) a ratio of a maximum plasma concentration of N-desethyloxybutynin to a maximum plasma concentration of oxybutynin (maximum plasma concentration of N-desethyloxybutynin/maximum plasma concentration of oxybutynin) at a time of single administration of the liquid topical preparation is 0.04 to 0.55;
(g) a ratio of an area under a plasma drug concentration-time curve of N-desethyloxybutynin from a time of administration to a final time of concentration measurement to an area under a plasma drug concentration-time curve of oxybutynin from a time of administration to a final time of concentration measurement in a single administration of the liquid topical preparation (area under a plasma drug concentration-time curve of N-desethyloxybutynin from a time of administration to a final time of concentration measurement/area under a plasma drug concentration-time curve of oxybutynin from a time of administration to a final time of concentration measurement) is 0.06 to 0.31;
(h) a ratio of a maximum plasma concentration of N-desethyloxybutynin to a maximum plasma concentration of oxybutynin (maximum plasma concentration of N-desethyloxybutynin/maximum plasma concentration of oxybutynin) when a steady state is reached by repeating administration of the liquid topical preparation once a day is 0.08 to 0.46;
(I) a ratio of an area under a plasma drug concentration-time curve of N-desethyloxybutynin in an administration interval to an area under a plasma drug concentration-time curve of oxybutynin in an administration interval (area under a plasma drug concentration-time curve of N-desethyloxybutynin in an administration interval/area under a plasma drug concentration-time curve of oxybutynin in an administration interval) when a steady state is reached by repeating administration of the liquid topical preparation is 0.15 to 0.65.

### EXAMPLES

### Test Example 1

Lotions were prepared according to the compositions in Table 1, and then 500 μL of each lotion was applied to the palms of subjects (4 subjects). Each subject spread the lotion evenly over both palms by rubbing the palms together, and then 3 minutes later, gave scores according to the degree of “stickiness” based on the following 3 stages.
0: No stickiness felt
1: Stickiness felt
2: Strong stickiness felt

Based on the mean value of the scores, the degree of stickiness was evaluated based on the following 3 stages.
◯: Mean value was less than 0.5
Δ: Mean value was 0.5 or more and less than 1.0
×: Mean value was 1.0 or more

TABLE 1

| Reference example | 1 | 2 | 3 | 4 |
|---|---|---|---|---|
| Oxybutynin hydrochloride | 8 | 10 | 15 | 20 |
| Sodium lactate | 2.3 | 2.8 | 4.3 | 5.7 |
| Ethanol | 40 | 40 | 40 | 40 |
| Purified water | 49.7 | 47.2 | 40.7 | 34.3 |
| Total | 100 | 100 | 100 | 100 |

Results are shown in Table 2. When the content of oxybutynin hydrochloride was 10 mass % or more, the subjects felt stickiness, and when the same was 15 mass % or more, the subjects felt strong stickiness.

TABLE 2

| Reference example | 1 | 2 | 3 | 4 |
|---|---|---|---|---|
| Mean value | 0.25 | 0.75 | 1.75 | 2 |
| Evaluation | ∘ | Δ | x | x |

### Test Example 2

Lotions were prepared according to the compositions in Table 3, and then the impulse value of each lotion was measured by the following method. It is indicated that the lower the impulse value, the lower the viscosity.

1) 50 μL of a lotion was placed in a 96-well plate, left to stand overnight at 32° C., and then dried.
2) A probe made of SUS (diameter: 5 mm) of a texture analyzer was brought into contact with the dried lotion.
3) The probe was ascended at the speed of 2 mm/second, the force applied thereto when the probe was moved away from the lotion was measured, and then the area under the curve was calculated as an impulse value (g second).

TABLE 3

| | Comparative example | | | | | | | | | | |
|---|---|---|---|---|---|---|---|---|---|---|---|
| | 1 | 2 | 3 | 4 | 5 | 6 | 7 | 8 | 9 | 10 | 11 |
| Oxybutynin | 20 | 20 | 20 | 20 | 20 | 20 | 20 | 20 | 20 | 20 | 20 |
| hydrochloride | | | | | | | | | | | |
| Sodium lactate | 5.7 | 5.7 | 5.7 | 5.7 | 5.7 | 5.7 | 5.7 | 5.7 | 5.7 | 5.7 | 5.7 |
| Eucalyptus oil | | 5 | | | | | | | | | |
| Dimethicone 350 | | | 5 | | | | | | | | |
| Lauryl alcohol | | | | 5 | | | | | | | |
| Oleyl alcohol | | | | | 5 | | | | | | |
| PEG200 | | | | | | 5 | | | | | |
| PEG400 | | | | | | | 5 | | | | |
| Monolaurate PEG | | | | | | | | 5 | | | |
| Lauromacrogol | | | | | | | | | 5 | | |
| POE oleyl ether | | | | | | | | | | 5 | |
| POE cetyl ether | | | | | | | | | | | 5 |
| Ethanol | 40 | 40 | 40 | 40 | 40 | 40 | 40 | 40 | 40 | 40 | 40 |
| Purified water | 34.3 | 29.3 | 29.3 | 29.3 | 29.3 | 29.3 | 29.3 | 29.3 | 29.3 | 29.3 | 29.3 |
| Total | 100 | 100 | 100 | 100 | 100 | 100 | 100 | 100 | 100 | 100 | 100 |
| | Example | | | | | | | | | | |
| | 1 | 2 | 3 | 16 | 17 | 18 | | | | | |
| Oxybutynin | 20 | 20 | 20 | 20 | 20 | 20 | | | | | |
| hydrochloride | | | | | | | | | | | |
| Sodium lactate | 5.7 | 5.7 | 5.7 | 5.7 | 5.7 | 5.7 | | | | | |
| Diisopropyl adipate | 5 | | | | | | | | | | |
| Diethyl sebacate | | 5 | | | | | | | | | |
| Diisopropyl | | | 5 | | | | | | | | |
| sebacate | | | | | | | | | | | |
| Diisobutyl adipate | | | | 5 | | | | | | | |
| Dimethyl succinate | | | | | 5 | | | | | | |
| Dibutyl phthalate | | | | | | 5 | | | | | |
| Ethanol | 40 | 40 | 40 | 40 | 40 | 40 | | | | | |
| Purified water | 29.3 | 29.3 | 293. | 29.3 | 29.3 | 29.3 | | | | | |
| Total | 100 | 100 | 100 | 100 | 100 | 100 | | | | | |

Results are shown in Table 4. The impulse value of each lotion is the mean value of three measurements. The lotion supplemented with lauryl alcohol, oleyl alcohol, lauromacrogol, diisopropyl adipate, diethyl sebacate, diisopropyl sebacate, diisobutyl adipate, dimethyl succinate or dibutyl phthalate, when dried, had a low impulse value compared with other lotions, which confirmed the decrease in viscosity.

TABLE 4

| | Comparative example | | | | | | | | | | |
|---|---|---|---|---|---|---|---|---|---|---|---|
| | 1 | 2 | 3 | 4 | 5 | 6 | 7 | 8 | 9 | 10 | 11 |
| Impulse | 6.4 | 4.8 | 8.4 | 1.4 | 1.2 | 4.9 | 5.0 | 5.3 | 1.2 | 3.6 | 3.5 |
| value | | | | | | | | | | | |
| | Example | | | | | | | | | | |
| | 1 | 2 | 3 | 16 | 17 | 18 | | | | | |
| Impulse | 0.5 | 1.5 | 1.3 | 0.91 | 0.39 | 1.3 | | | | | |
| value | | | | | | | | | | | |

### Test Example 3

Lotions were prepared according to the compositions in Table 5, and then 300 μL of each lotion was applied to the palms of subjects (3 subjects). In Comparative examples 1, 4, 5 and 9 as well as Examples 1 to 3, the same lotions as in test example 2 were used. Each subject spread the lotion evenly over both palms by rubbing the palms together, and then 3 minutes later, gave scores according to the degree of “stickiness” based on the following 4 stages.
0: No stickiness felt
1: Slight stickiness felt
2: Stickiness felt
3: Strong stickiness felt

Based on the mean value of the scores, the degree of stickiness was evaluated based on the following 5 stages.
⊚: Mean value was less than 0.1
◯: Mean value was 0.1 or more and less than 1.0
Δ: Mean value was 1.0 or more and less than 2.0
×: Mean value was 2.0 or more and less than 3.0
××: Mean value was 3.0 or more

TABLE 5

| | Comparative example | | | | | | | | |
|---|---|---|---|---|---|---|---|---|---|
| | 1 | 4 | 5 | 9 | | | | | |
| Oxybutynin | 20 | 20 | 20 | 20 | | | | | |
| hydrochloride | | | | | | | | | |
| Sodium lactate | 5.7 | 5.7 | 5.7 | 5.7 | | | | | |
| Lauryl alcohol | | 5 | | | | | | | |
| Oleyl alcohol | | | 5 | | | | | | |
| Lauromacrogol | | | | 5 | | | | | |
| Ethanol | 40 | 40 | 40 | 40 | | | | | |
| Purified water | 34.3 | 34.3 | 34.3 | 34.3 | | | | | |
| Total | 100 | 100 | 100 | 100 | | | | | |
| Oxybutynin | 1:0 | 1:0 | 1:0 | 1:0 | | | | | |
| hydrochloride: | | | | | | | | | |
| dicarboxylic | | | | | | | | | |
| acid ester | | | | | | | | | |
| | Example | | | | | | | | |
| | 1 | 2 | 3 | 4 | 5 | 6 | 7 | 8 | 9 |
| Oxybutynin | 20 | 20 | 20 | 20 | 20 | 20 | 20 | 20 | 20 |
| hydrochloride | | | | | | | | | |
| Sodium lactate | 5.7 | 5.7 | 5.7 | 5.7 | 5.7 | 5.7 | 5.7 | 5.7 | 5.7 |
| Diisopropyl | 5 | | | 8 | | 10 | | 15 | |
| adipate | | | | | | | | | |
| Diethyl | | 5 | | | 8 | | 10 | | 15 |
| sebacate | | | | | | | | | |
| Diisopropyl | | | 5 | | | | | | |
| sebacate | | | | | | | | | |
| Ethanol | 40 | 40 | 40 | 40 | 40 | 40 | 40 | 40 | 40 |
| Purified water | 29.3 | 29.3 | 29.3 | 26.3 | 26.3 | 24.3 | 24.3 | 19.3 | 19.3 |
| Total | 100 | 100 | 100 | 100 | 100 | 100 | 100 | 100 | 100 |
| Oxybutynin | | | | | | | | | |
| hydrochloride: | 1:0.25 | 1:0.25 | 1:0.25 | 1:0.4 | 1:0.4 | 1:0.5 | 1:0.5 | 1:0.75 | 1:0.75 |
| dicarboxylic | | | | | | | | | |
| acid ester | | | | | | | | | |
| | Comparative | | | | Comparative | | | | |
| | example | Example | | | example | Example | | | |
| | 12 | 10 | 11 | 12 | 13 | 13 | 14 | 15 | |
| Oxybutynin | 15 | 15 | 15 | 15 | 10 | 10 | 10 | 10 | |
| hydrochloride | | | | | | | | | |
| Sodium lactate | 4.28 | 4.28 | 4.28 | 4.28 | 2.85 | 2.85 | 2.85 | 2.85 | |
| Diisopropyl | | 3.75 | 5 | 11.25 | | 2.5 | 5 | 7.5 | |
| adipate | | | | | | | | | |
| Ethanol | 40 | 40 | 40 | 40 | 40 | 40 | 40 | 40 | |
| Purified water | 40.72 | 36.97 | 35.72 | 29.47 | 47.15 | 44.65 | 42.15 | 39.65 | |
| Total | 100 | 100 | 100 | 100 | 100 | 100 | 100 | 100 | |
| Oxybutynin | 1:0 | 1:0.25 | 1:0.33 | 1:0.75 | 1:0 | 1:0.25 | 1:□0.5 | 1:0.75 | |
| hydrochloride: | | | | | | | | | |
| dicarboxylic | | | | | | | | | |
| acid ester | | | | | | | | | |

Results are shown in Table 6. The lotion supplemented with diisopropyl adipate, diethyl sebacate or diisopropyl sebacate was confirmed to have lowered stickiness compared with lotions comprising no dicarboxylic acid ester.

TABLE 6

| | Comparative example | | | | | | | | |
|---|---|---|---|---|---|---|---|---|---|
| | 1 | 4 | 5 | 9 | | | | | |
| Mean | 3 | 2 | 2 | 2 | | | | | |
| value | | | | | | | | | |
| Evaluation | ×× | × | × | × | | | | | |
| | Example | | | | | | | | |
| | 1 | 2 | 3 | 4 | 5 | 6 | 7 | 8 | 9 |
| Mean | 1 | 0.67 | 0.33 | 0 | 0 | 0 | 0 | 0 | 0.33 |
| value | | | | | | | | | |
| Evaluation | Δ | ○ | ○ | ⊚ | ⊚ | ⊚ | ⊚ | ⊚ | ○ |
| | Comparative | | | | Comparative | | | | |
| | example | Example | | | example | Example | | | |
| | 12 | 10 | 11 | 12 | 13 | 13 | 14 | 15 | |
| Mean | 2.33 | 0.67 | 0.67 | 0 | 1.33 | 0.67 | 0.33 | 0 | |
| value | | | | | | | | | |
| Evaluation | × | ○ | ○ | ⊚ | Δ | ○ | ○ | ⊚ | |

### Test Example 4

Using the lotions of Examples 1 to 9 and Comparative example 1 in test examples 2 and 3, the preservation stability of oxybutynin was evaluated by the following method.

1) 100 μL of each lotion was measured and weighed.
2) 50 mL of mobile phase was added and mixed therewith, and then the oxybutynin concentration of each lotion was measured by high-performance liquid chromatography (HPLC). HPLC conditions are as follows.
Mobile phase: 0.1 w/w % aqueous phosphoric acid solution (containing 0.5 w/v % sodium dodecyl sulfate):acetonitrile=45:55(v/v)
Flow rate: 1.5 mL/minute

### Column: TSK gel ODS-80 Ts (Tosoh Corporation)

Retention time: 10 minutes
3) After the lotion was left to stand at 60° C. for 2 weeks, the oxybutynin content (%) per solution weight, relative to the theoretical value, was compared with the initial oxybutynin content (%) relative to the theoretical value. When the oxybutynin content (%) after the lotion was left to stand at 60° C. for 2 weeks was 97.5% or more of the initial oxybutynin content (%), the preservation stability of the oxybutynin was evaluated as good.

Results are shown in Table 7. In the lotion supplemented with diisopropyl adipate, diethyl sebacate or diisopropyl sebacate, oxybutynin hydrochloride was preserved well even after the lotion was left to stand under an environment at 60° C. for 2 weeks. Moreover, in Comparative example 1 wherein no dicarboxylic acid ester was added, oxybutynin hydrochloride was preserved well.

TABLE 7

| | | | | After left to |
|---|---|---|---|---|
| | | Initial | After left | stand/initial |
| | | value | to stand | value (%) |
| Example | 1 | 101.1 | 101.3 | 100.2 |
| | 2 | 102.2 | 100.6 | 98.4 |
| | 3 | 101.2 | 100.4 | 99.1 |
| | 4 | 100.3 | 100.2 | 99.9 |
| | 5 | 100.3 | 99.8 | 99.5 |
| | 6 | 100.4 | 100.0 | 99.6 |
| | 7 | 101.3 | 100.9 | 99.6 |
| | 8 | 102.1 | 100.9 | 98.8 |
| | 9 | 101.7 | 100.7 | 99.0 |
| Comparative | 1 | 100.8 | 99.7 | 98.9 |
| example | | | | |

### Test Example 5

Using the lotions of Comparative example 1 and Example 4 in test example 3, the skin permeability of oxybutynin was determined by the following method.

1) 5 μL of each lotion was applied to an area of 3 cm² on the dermatomed human skin surface.
2) After several seconds of drying, the skin was set in Franz Cell so that the skin dermis side was the receptor layer side. As the receptor layer, physiological saline was used. At the time points, 4, 8, 12, 16, 20 and 24 hours after setting of the skin, the receptor solution was sampled. To 0.5 mL of the sampled solution, 0.5 mL of acetonitrile was added, the mixture was stirred, and then centrifugation was performed for deproteinization, thereby preparing a test solution.
3) The oxybutynin concentration in the test solution was measured by HPLC under the same conditions as those in test example 4.
4) The skin permeation rate of oxybutynin per hour was calculated from the thus obtained measurement, and the maximum value was designated as Jmax (μg/cm²/h). Furthermore, the cumulative amount (μg/cm²) permeated in 24 hours was found.

Results are shown in Table 8. The lotion supplemented with diisopropyl adipate exerted the same degree of skin permeability as that of lotions comprising no dicarboxylic acid ester.

TABLE 8

| | | Cumulative | |
|---|---|---|---|
| | Jₘₐₓ | amount_24 h | Availability |
| | (ug/cm²/h) | (ug/cm²) | (%) |
| Comparative | 0.58 | 9.4 | 2.8 |
| example 1 | | | |
| Example 4 | 0.50 | 8.4 | 2.5 |

### Test Example 6

Lotions were prepared according to the compositions in Table 9, and visually confirmed for the state of dissolution. Furthermore, the lotions were applied to porcine skin, and then the amounts of oxybutynin accumulated in hair follicles were measured by the following method.

1) 20 μL of a lotion was applied to 5 cm² of lightly shaved porcine skin.
Number of pigs: n=3.
2) After 6 hours, the skin surface was cleaned with ethanol for disinfection, and washed with a stream of phosphate buffer, thereby removing oxybutynin that had adhered to the skin surface.
3) A hair follicle portion of 20 hairs was collected from the skin.
4) Oxybutynin was extracted from the hair follicles using 1 mL of an extracting liquid. As the extracting liquid, the mobile phase of test example 4 was used.
5) Oxybutynin concentration was measured by HPLC. HPLC conditions are the same as those in test example 4.

TABLE 9

| Composition | 1 | 2 | 3 | 4 | 5 | 6 | 7 | 8 | 9 | 10 |
|---|---|---|---|---|---|---|---|---|---|---|
| Oxybutynin | 4.54 | — | | | | | | | | |
| Oxybutynin | — | 5 | 5 | 5 | 5 | 5 | 5 | 5 | 5 | 5 |
| hydrochloride | | | | | | | | | | |
| Sodium hydroxide | | | 0.51 | | | | | | | |
| Disodium | | | | 0.9 | | | | | | |
| hydrogenphosphate | | | | | | | | | | |
| Sodium lactate | | | | | 1.42 | | | | | |
| Sodium acetate | | | | | | 1.04 | | | | |
| Disodium fumarate | | | | | | | 1.02 | | | |
| Trisodium citrate | | | | | | | | 1.09 | | |
| Sodium benzoate | | | | | | | | | 1.83 | |
| Disodium | | | | | | | | | | 2.92 |
| tartrate·dihydrate | | | | | | | | | | |
| Ethanol | 40 | 40 | 40 | 40 | 40 | 40 | 40 | 40 | 40 | 40 |
| Others | 0.2 | 0.2 | 0.2 | 0.2 | 0.2 | 0.2 | 0.2 | 0.2 | 0.2 | 0.2 |
| Purified water | 55.26 | 54.8 | 52.49 | 53.9 | 53.38 | 58.76 | 53.78 | 53.71 | 52.97 | 51.88 |
| Total | 100 | 100 | 100 | 100 | 100 | 100 | 100 | 100 | 100 | 100 |

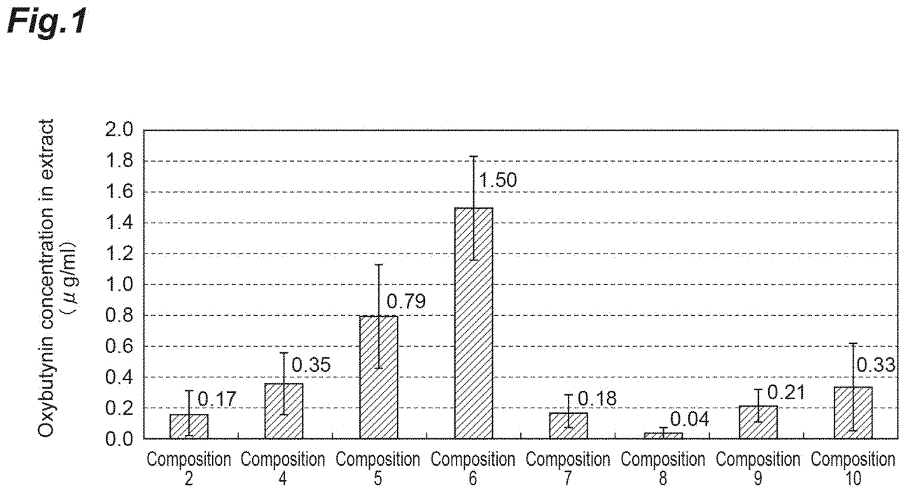

Results are shown in Table 10 and FIG. 1. With the lotion comprising phosphate, lactate, acetate or tartrate, accumulation of oxybutynin in hair follicles was high compared to lotions comprising none of these salts.

TABLE 10

| Composition | 1 | 2 | 3 | 4 | 5 | 6 | 7 | 8 | 9 | 10 |
|---|---|---|---|---|---|---|---|---|---|---|
| State of dissolution | × | × | × | ○ | ○ | ○ | ○ | ○ | ○ | ○ |

### Test Example 7

Lotions were prepared according to the compositions in Table 11. The lotions were determined for the effect of suppressing sweating by a pilocarpine-induced sweat test. Moreover, in a manner similar to that in test example 6, the lotions were applied to porcine skin, and then oxybutynin concentrations were measured.

The pilocarpine-induced sweat test was conducted by the following method.

1) A lotion was diluted 12-fold with a 40 mass % aqueous ethanol solution.
2) 10 μL or 15 μL of the lotion was applied to about 0.5 cm² of a mouse footpad. Number of mice: n=5 to 6.
3) After 4 hours, iodine and a starch solution were applied to the footpad under anesthesia.
4) Pilocarpine was intradermally administered at 5 μg/foot.
5) After 5 minutes, the number of black spots resulting from the iodostarch reaction was counted.

TABLE 11

| Composition | 11 | 12 | 13 | 14 | 15 |
|---|---|---|---|---|---|
| Oxybutynin | 0 | 1 | 2.5 | 5 | 10 |
| hydrochloride | | | | | |
| Lactic acid | 2.29 | | | | |
| Sodium chloride | 1.48 | | | | |
| Sodium lactate | | 0.28 | 0.71 | 1.42 | 2.84 |
| Ethanol | 40 | 40 | 40 | 40 | 40 |
| Others | 2 | 2 | 2 | 2 | 2 |
| Purified water | 54.23 | 56.72 | 54.79 | 51.58 | 45.16 |
| Total | 100 | 100 | 100 | 100 | 100 |

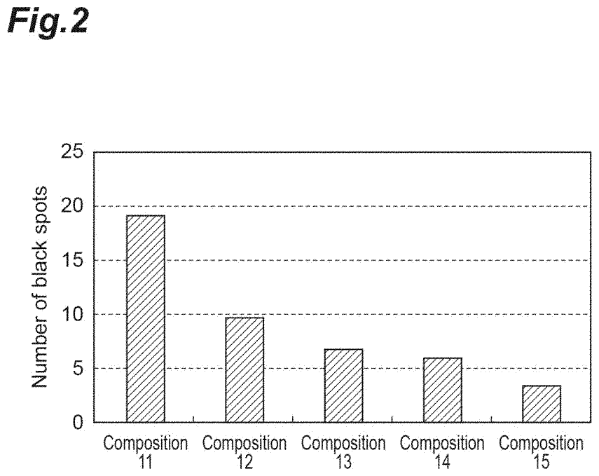

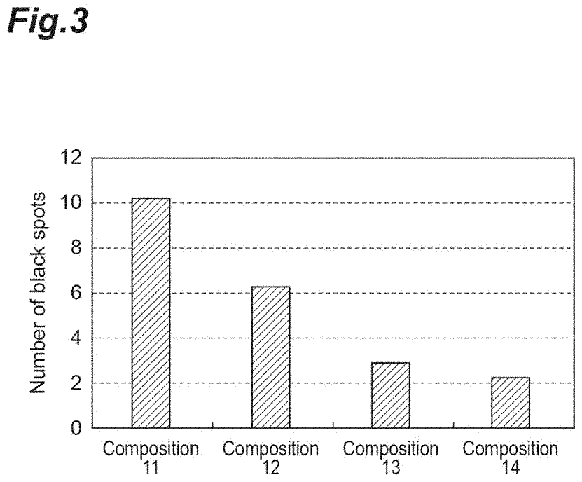

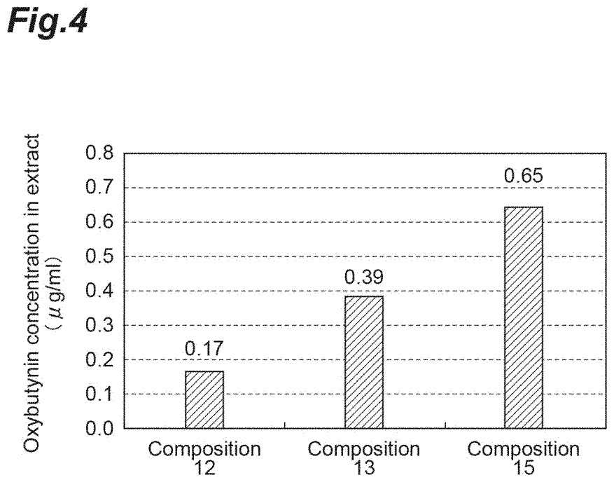

Results are shown in FIG. 2 to FIG. 4. FIG. 2 shows the results of the pilocarpine-induced sweat test when the amount of each lotion applied was 10 μL, and FIG. 3 shows the results of the pilocarpine-induced sweat test when the amount of each lotion applied was 15 μL. It was confirmed that the lotions' effect of suppressing sweating was oxybutynin concentration-dependent. It was also confirmed that the amounts of oxybutynin accumulated in hair follicles were oxybutynin concentration-dependent.

1. Production of Liquid Topical Preparation

Liquid topical preparations 1 to 3 (lotion preparations) were produced by homogeneously mixing the components shown in Table 12, and filled in containers. The densities of the obtained liquid topical preparations were 963 mg/cm³ for liquid topical preparation 1, 945 mg/cm³ for liquid topical preparation 2, and 931 mg/cm³ for liquid topical preparation 3.

TABLE 12

| Liquid topical preparation | 1 | 2 | 3 |
|---|---|---|---|
| Oxybutynin hydrochloride | 20 | 5 | 0 |
| Sodium lactate | 6 | 1.5 | 0 |
| Diisopropyl adipate | 8 | 0 | 0 |
| Anhydrous ethanol | 40 | 40 | 40 |
| Purified water | 26 | 53.5 | 60 |
| Total | 100 | 100 | 100 |

2. Administration of Liquid Topical Preparation

182 hyperhidrosis patients were divided into three groups, and 500 μL of each of liquid topical preparation 1 to 3 (containing oxybutynin hydrochloride 96 mg, 24 mg, and 0 mg, respectively) was applied to both palms of each of the patients once a day at bedtime. The administration of the liquid topical preparation was repeated for 8 weeks.

3. Measurement of Sweat Amount

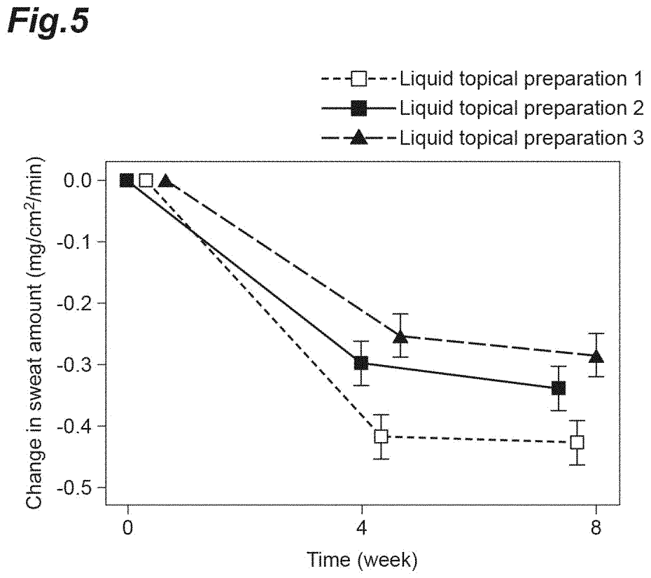

Before administration, and at 4 weeks and 8 weeks from the start of administration, the amount of sweat of the palms of the patients was measured using a sweat amount measuring device (ventilation capsule type perspiration meter SKN-2000M; manufactured by Nishizawa Electric Meter Manufacturing Co., Ltd.). The amount of change in the sweat amount of each group was calculated as (amount of change in sweat amount)=(amount of sweat after administration of liquid topical preparation)−(amount of sweat before administration of liquid topical preparation). The results of the change in the amount of sweat are shown in FIG. 5. As a result of statistical analysis (Mixed-effects Model Repeated Measures (MMRM)), the amount of sweat was significantly lower in liquid topical preparation 1-administered group than in liquid topical preparation 3-administered group at 4 weeks (p=0.0015) and 8 weeks (p=0.0046) after the start of administration.

4. Evaluation of Subjective Symptoms of Patients

Before the administration and at 2 weeks, 4 weeks, 6 weeks and 8 weeks after the start of the administration, the patients themselves were asked to evaluate the state of sweat based on the following criteria

### (Hyperhidrosis Disease Severity Scale; HDSS).

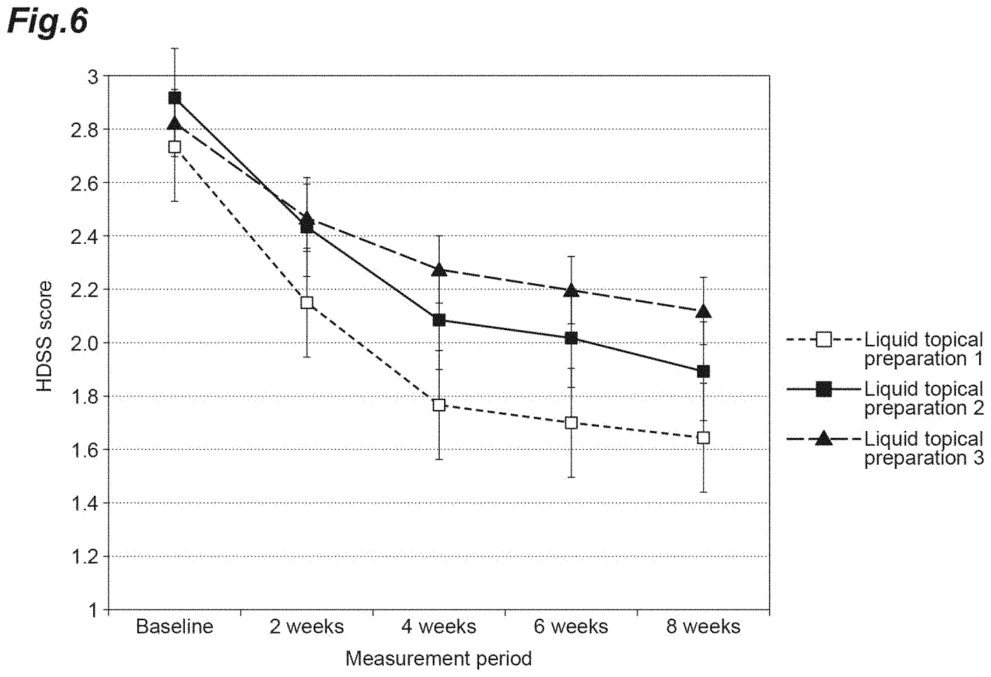

Grade 1: My sweating is never noticeable and never interferes with my daily activities.
Grade 2: My sweating is tolerable but sometimes interferes with my daily activities.
Grade 3: My sweating is barely tolerable and frequently interferes with my daily activities.
Grade 4: My sweating is intolerable and always interferes with my daily activities.
The results of the HDSS scores are shown in FIG. 6.

5. Measurement of Plasma Concentrations of Oxybutynin and N-Desethyloxybutynin

Blood was collected from the patients at 2 weeks, 4 weeks, 6 weeks, and 8 weeks after the start of administration, and the collected samples were analyzed using a liquid chromatography tandem mass spectrometry system (LC-MS/MS) to measure the mean plasma concentrations of oxybutynin and N-desethyloxybutynin. The LC-MS/MS apparatuses used were high-performance liquid chromatograph Prominence UFLC System (manufactured by Shimadzu Corporation) and a mass spectrometer QTRAP5500 (manufactured by AB Sciex Corporation), or high-performance liquid chromatograph Nexera UHPLC System (manufactured by Shimadzu Corporation) and a mass spectrometer Triple Quad 5500 (manufactured by AB Sciex Corporation).

The mean plasma concentrations (ng/mL) of the patients who received liquid topical preparation 1 are as follows in Table 13.

TABLE 13

| | | | |
|---|---|---|---|
| 2 w | 4 w | 6 w | 8 w |

Oxybutynin

| | | | | |
|---|---|---|---|---|
| Mean | 20.2 | 20.3 | 20.5 | 17.7 |
| Standard deviation | 21.7 | 19.8 | 19.4 | 16.3 |

N-Desethyloxybutynin

| | | | | |
|---|---|---|---|---|
| Mean | 6.10 | 6.41 | 5.52 | 5.36 |
| Standard deviation | 5.83 | 6.56 | 4.85 | 5.06 |

Oxybutynin/N-Desethyloxybutynin

| | | | | |
|---|---|---|---|---|
| Ratio | 3.31 | 3.17 | 3.71 | 3.30 |

The mean plasma concentrations (ng/mL) of the patients who received liquid topical preparation 2 are as follows in Table 14.

TABLE 14

| | | | |
|---|---|---|---|
| 2 w | 4 w | 6 w | 8 w |

Oxybutynin

| | | | | |
|---|---|---|---|---|
| Mean | 9.96 | 11.2 | 6.80 | 6.78 |
| Standard deviation | 10.99 | 17.3 | 7.98 | 9.15 |

N-Desethyloxybutynin

| | | | | |
|---|---|---|---|---|
| Mean | 2.73 | 2.82 | 1.86 | 1.94 |
| Standard deviation | 2.71 | 2.83 | 1.65 | 2.45 |

Oxybutynin/N-Desethyloxybutynin

| | | | | |
|---|---|---|---|---|
| Ratio | 3.65 | 3.97 | 3.66 | 3.49 |

6. Pharmacokinetic Test of Liquid Topical Preparation

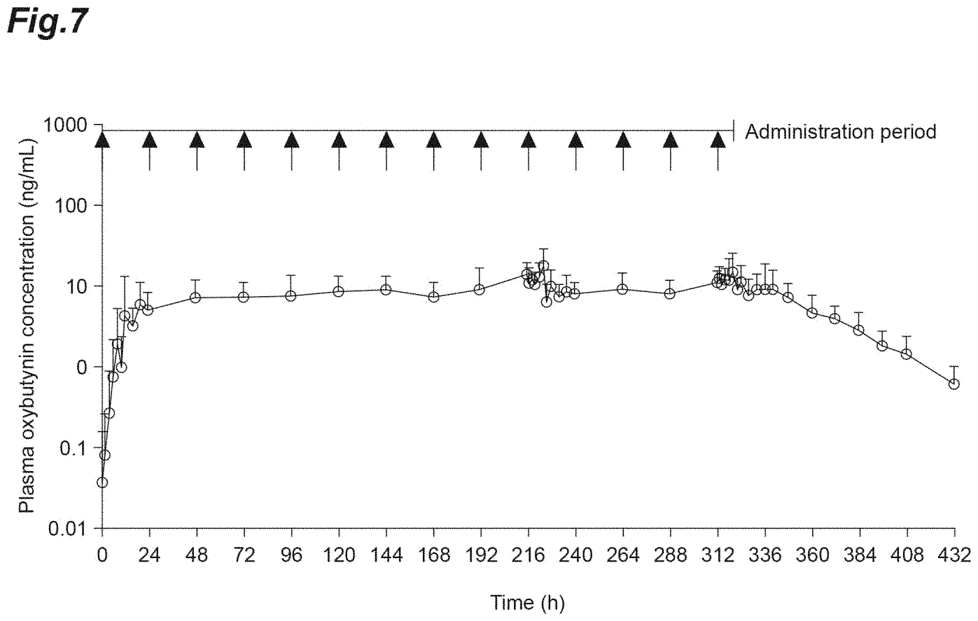

To 18 healthy adult males, 500 μL of liquid topical preparation 1 was applied to the both palms once a day for 8 hours for 14 days. Blood was collected immediately before administration of liquid topical preparation 1 and until 432 hours after the start of administration, and the collected samples were analyzed using a liquid chromatography tandem mass spectrometry system (LC-MS/MS) to measure the plasma concentrations of oxybutynin and N-desethyloxybutynin. The results of plasma concentration profile of oxybutynin are shown in FIG. 7. The LC-MS/MS apparatus used was the same as that used in the measurement of item 5 above.

The following pharmacokinetic parameters of liquid topical preparation 1 were calculated based on the measured plasma concentrations. The results for oxybutynin are shown in Table 15 and those for N-desethyloxybutynin are shown in Table 16.

C₂₄: plasma concentration at 24 hours after each time point
Cₘₐₓ: maximum plasma concentration during 24 hours from each time point
Tₘₐₓ: time required to reach the maximum plasma concentration
AUC₀₋₂₄: area under the plasma drug concentration-time curve from each time point to 24 hours later
Cₘᵢₙ: minimum plasma concentration during 24 hours from each time point
Cₐᵥ: mean plasma concentration for 24 hours from each time point
t_{1/2}: time required for the drug concentration to be halved (half-life) after liquid topical preparation applied on day 14 was removed (8 hours after application) (also referred to as “after final removal”)
CL/F: apparent clearance calculated from AUC₀₋₂₄ for routes of administration other than intravenous

TABLE 15

| | C₂₄ | | | Cₘₐₓ | | | tₘₐₓ | | | |
|---|---|---|---|---|---|---|---|---|---|---|
| | (ng/mL) | | | (ng/mL) | | | (h) | | | |
| Administration | 1st | 10th | 14th | 1st | 10th | 14th | 1st | 10th | 14th | |
| Case | 18 | 18 | 18 | 18 | 18 | 18 | 18 | 18 | 18 | |
| Mean | 4.97 | 7.74 | 9.14 | 8.11 | 18.6 | 17.5 | 20.2 | 8.1 | 8.5 | |
| S.D. | 3.04 | 3.15 | 9.05 | 8.55 | 9.44 | 8.98 | 4.0 | 4.9 | 4.1 | |
| C.V. (%) | 61.2 | 40.6 | 99.0 | 105.4 | 50.8 | 51.3 | 19.7 | 60.9 | 48.1 | |
| Median | 4.68 | 7.65 | 6.26 | 4.88 | 18.4 | 15.0 | 20.0 | 8.0 | 8.0 | |
| MM. | 0.730 | 2.08 | 3.02 | 0.814 | 7.59 | 5.92 | 12 | 1 | 1 | |
| Max. | 11.8 | 15.6 | 43.4 | 38.9 | 42.7 | 45.5 | 24 | 20 | 20 | |
| Mode | — | — | — | — | — | — | 20, 24 | 8 | 8 | |
| | | | | | | | | t_{1/2} | | |
| | | | | | | | | (h) | | |
| | AUC₀₋₂₄ | | | Cₘᵢₙ | | Cₐᵥ | | after | CL/F | |
| | (ng·h/mL) | | | (ng/mL) | | (ng/mL) | | final | (L/h) | |
| Administration | 1st | 10th | 14th | 10th | 14th | 10th | 14th | removal | 10th | 14th |
| Case | 18 | 18 | 18 | 18 | 18 | 18 | 18 | 17 | 18 | 18 |
| Mean | 64.8 | 230 | 241 | 4.94 | 5.64 | 9.58 | 10.1 | 27.2 | 443 | 437 |
| S.D. | 64.2 | 84.7 | 115 | 2.40 | 3.29 | 3.53 | 4.80 | 18.0 | 195 | 209 |
| C.V. (%) | 99.1 | 36.8 | 47.8 | 48.7 | 58.4 | 36.8 | 47.8 | 66.2 | 44.0 | 47.8 |
| Median | 41.6 | 208 | 224 | 4.43 | 5.59 | 8.67 | 9.34 | 23.6 | 424 | 394 |
| Min. | 9.79 | 86.6 | 79.6 | 2.08 | 2.09 | 3.61 | 3.32 | 10.2 | 221 | 145 |
| Max. | 291 | 397 | 606 | 9.87 | 16.1 | 16.5 | 25.2 | 89.4 | 1010 | 1100 |
| Mode | — | — | — | — | — | — | — | — | — | — |

TABLE 16

| | C₂₄ | | | Cₘₐₓ | | | tₘₐₓ | | |
|---|---|---|---|---|---|---|---|---|---|
| | (ng/mL) | | | (ng/mL) | | | (h) | | |
| Administration | 1st | 10th | 14th | 1st | 10th | 14th | 1st | 10th | 14th |
| Case | 18 | 18 | 18 | 18 | 18 | 18 | 18 | 18 | 18 |
| Mean | 0.942 | 2.61 | 3.01 | 0.945 | 2.98 | 3.27 | 23.2 | 7.6 | 11.2 |
| S.D. | 0.494 | 1.30 | 1.78 | 0.493 | 1.57 | 1.83 | 3.3 | 9.5 | 10.3 |
| C.V.( %) | 52.4 | 49.8 | 59.1 | 52.1 | 52.8 | 55.9 | 14.2 | 125.3 | 91.6 |
| Median | 0.763 | 2.26 | 2.37 | 0.793 | 2.65 | 2.44 | 24.0 | 1.0 | 9.0 |
| Min. | 0.365 | 1.08 | 1.19 | 0.365 | 1.13 | 1.28 | 10 | 1 | 1 |
| Max. | 1.76 | 5.62 | 7.34 | 1.76 | 6.93 | 7.34 | 24 | 24 | 24 |
| Mode | — | — | — | — | — | — | 24 | 1 | 1, 24 |
| | | | | | | | | t_{1/2} (h) | |
| | AUC₀₋₂₄ | | | Cₘᵢₙ | | Cₐᵥ | | after | |
| | (ng·h/mL) | | | (ng/mL) | | (ng/mL) | | final | |
| Administration | 1st | 10th | 14th | 10th | 14th | 10th | 14th | removal | |
| Case | 18 | 18 | 18 | 18 | 18 | 18 | 18 | 18 | |
| Mean | 8.06 | 55.3 | 62.0 | 1.83 | 2.16 | 2.30 | 2.59 | 27.4 | |
| S.D. | 5.42 | 26.6 | 32.5 | 0.865 | 1.21 | 1.11 | 1.35 | 4.48 | |
| C.V. (%) | 67.3 | 48.2 | 52.3 | 47.3 | 56.0 | 48.2 | 52.3 | 16.4 | |
| Median | 5.94 | 50.1 | 46.6 | 1.71 | 1.61 | 2.09 | 1.94 | 26.4 | |
| Min. | 2.36 | 24.4 | 26.5 | 0.857 | 0.977 | 1.02 | 1.10 | 20.7 | |
| Max. | 21.0 | 114 | 137 | 3.68 | 5.21 | 4.73 | 5.71 | 37.4 | |
| Mode | — | — | — | — | — | — | — | — | |

The molecular weight corrected N-desethyloxybutynin/oxybutynin ratios of the above pharmaceutical parameters (C₂₄, Cₘₐₓ and AUC₀₋₂₄) are shown in Table 17.

TABLE 17

| | C₂₄ | | | Cₘₐₓ | | | AUC₀₋₂₄ | | |
|---|---|---|---|---|---|---|---|---|---|
| Administration | 1st | 10th | 14th | 1st | 10th | 14th | 1st | 10th | 14th |
| Case | 18 | 18 | 18 | 18 | 18 | 18 | 18 | 18 | 18 |
| Mean | 0.246 | 0.389 | 0.453 | 0.174 | 0.184 | 0.213 | 0.163 | 0.266 | 0.291 |
| S.D. | 0.128 | 0.168 | 0.305 | 0.102 | 0.064 | 0.097 | 0.067 | 0.095 | 0.121 |
| C.V. (%) | 52.1 | 43.1 | 67.4 | 58.8 | 35.1 | 45.6 | 41.0 | 35.8 | 41.6 |
| Median | 0.208 | 0.337 | 0.395 | 0.148 | 0.193 | 0.187 | 0.140 | 0.232 | 0.242 |
| Min. | 0.104 | 0.193 | 0.184 | 0.049 | 0.084 | 0.112 | 0.062 | 0.160 | 0.194 |
| Max. | 0.586 | 0.783 | 1.498 | 0.525 | 0.333 | 0.459 | 0.303 | 0.552 | 0.607 |

Cumulative coefficients of plasma concentrations of oxybutynin and N-desethyloxybutynin (R_{obs}: Cₘₐₓ or AUC₀₋₂₄ at 10th and 14th administration/Cₘₐₓ or AUC₀₋₂₄ at 1st administration) were calculated. The results for oxybutynin are shown in Table 18 and those for N-desethyloxybutynin are shown in Table 19.

TABLE 18

| R_{obs} | |
|---|---|
| Cₘₐₓ | AUC₀₋₂₄ |

| Administration | 10th | 14th | 10th | 14th |
|---|---|---|---|---|
| Case | 18 | 18 | 18 | 18 |
| Mean | 3.555 | 3.628 | 5.528 | 6.444 |
| S.D. | 2.423 | 3.062 | 3.465 | 6.861 |
| C.V.(%) | 68.2 | 84.4 | 62.7 | 106.5 |
| Median | 2.889 | 2.866 | 4.717 | 5.002 |
| Min. | 0.542 | 0.509 | 1.053 | 1.013 |
| Max. | 9.324 | 13.422 | 14.722 | 31.909 |

TABLE 19

| R_{obs} | |
|---|---|
| Cₘₐₓ | AUC₀₋₂₄ |

| Administration | 10th | 14th | 10th | 14th |
|---|---|---|---|---|
| Case | 18 | 18 | 18 | 18 |
| Mean | 3.434 | 4.113 | 9.000 | 10.752 |
| S.D. | 1.327 | 3.351 | 5.118 | 9.310 |
| C.V.(%) | 38.6 | 81.5 | 56.9 | 86.6 |
| Median | 3.239 | 3.495 | 7.515 | 8.846 |
| Min. | 1.795 | 1.448 | 2.088 | 2.203 |
| Max. | 6.301 | 16.682 | 21.198 | 43.868 |

As shown in Table 18, the cumulative coefficients (mean values) of plasma oxybutynin concentrations were 3.555 and 3.628 at the 10th administration and the 14th administration for Cₘₐₓ, and 5.528 and 6.444 at the 10th administration and the 14th administration for AUC₀₋₂₄. In addition, as shown in Table 17, the cumulative coefficients (mean values) of the plasma N-desethyloxybutynin concentrations were 3.434 and 4.113 at the 10th administration and the 14th administration for Cₘₐₓ, and 9.000 and 10.752 at the 10th administration and the 14th administration for AUC₀₋₂₄.

The cumulative coefficients of plasma oxybutynin concentration were comparable between the 10th and 14th administrations for both pharmacokinetic parameters and it was considered to have reached a steady state. Similarly, the cumulative coefficients of the plasma N-desethyloxybutynin concentration were approximately the same in both pharmacokinetic parameters at the 10th and 14th administrations, and it was considered to have reached a steady state.

It is to be understood that the methods for treating hyperhidrosis are not limited to the specific embodiments described above, but encompass any and all embodiments within the scope of the generic language of the following claims enabled by the embodiments described herein, or otherwise shown in the drawings or described above in terms sufficient to enable one of ordinary skill in the art to make and use the claimed subject matter.

1. A method for treating hyperhidrosis comprising administering to a patient in need thereof, a liquid topical preparation, comprising:
water; and
oxybutynin or a pharmaceutically acceptable salt thereof;
wherein the oxybutynin or pharmaceutically acceptable salt thereof is present in a range of from 10 mass % to 20 mass % based on a total mass of the liquid topical preparation.

2. The method for treating hyperhidrosis of claim 1, wherein the method comprises administering the liquid topical preparation once daily.

3. The method for treating hyperhidrosis of claim 1, wherein the method comprises administering the liquid topical preparation to both of the patient's palms.

4. The method for treating hyperhidrosis of claim 1, wherein the oxybutynin or pharmaceutically acceptable salt thereof comprises oxybutynin hydrochloride.

5. The method for treating hyperhidrosis of claim 1, wherein the oxybutynin or pharmaceutically acceptable salt thereof is present at about 20 mass % based on a total mass of the liquid topical preparation.

6. The method for treating hyperhidrosis of claim 1, wherein the liquid topical preparation is administered in the form of a lotion.

7. The method for treating hyperhidrosis of claim 1, wherein the liquid topical preparation further comprises at least one ingredient to enhance accumulation of the oxybutynin or pharmaceutically acceptable salt thereof in the patient.

8. The method for treating hyperhidrosis of claim 7, wherein the at least one ingredient to enhance accumulation comprises at least one salt selected from the group consisting of lactate, tartrate, acetate, and phosphate.

9. The method for treating hyperhidrosis of claim 8, wherein the at least one salt includes sodium lactate.

10. The method for treating hyperhidrosis of claim 8, wherein the liquid topical formulation further comprises ethanol.

11. The method for treating hyperhidrosis of claim 5,
wherein the oxybutynin or pharmaceutically acceptable salt thereof comprises oxybutynin hydrochloride;
and wherein the liquid topical preparation further comprises sodium lactate and ethanol.

12. The method for treating hyperhidrosis of claim 11, wherein the liquid topical preparation is administered in the form of a lotion.

13. The method for treating hyperhidrosis of claim 11, wherein the liquid topical preparation is administered once daily to both of the patient's palms, for at least 10 days.

14. The method for treating hyperhidrosis of claim 13, wherein the liquid topical preparation is administered for at least 4 weeks.

15. The method for treating hyperhidrosis of claim 13, wherein the administration reduces sweat produced by the patient's palm by at least about 0.4 mg/cm²/minute.
